(19) United States
(12) Patent Application Publication
(10) Pub. No.: US 2003/0114876 A1
(43) Pub. Date: Jun. 19, 2003

(54) Device for use by brain operations

(76) Inventors: Eigil Samset, Oslo (NO); Henry Hirschberg, Oslo (NO)

Correspondence Address: Nixon & Vanderhye, 8th Floor, 1100 North Glebe Road, Arlington, VA, 22201-4714, US

(21) Appl. No.: 10/169,482

(22) PCT Filed: Jan. 3, 2001
(86) PCT No.: PCT/NO01/00001

(30) Foreign Application Priority Data
Jan. 6, 2000 (NO) 20000067

(51) Int. Cl. A61B 17/32

(52) U.S. Cl. 606/172

(57) ABSTRACT

Tool for use by brain operation comprising a guiding/support-tool (1) provided for mounting on the head of a patient over an operation aperture as for example a probe may be inserted into the brain of the patient, along with system to determine the depth of the insertion and coordinate of the insertion tool for use by brain operation. Said tool (1) having fastening means for a pointing device, which is connected with an image processing unit for calculation of the insertion depth of the probe, the coordinate and direction of the probe/insertion tool may be ovelaid the visualized image respectively images of the brain. Further said tool (1) has adjusting means (7, 21) for adjusting the tool (1) in desired direction of the probe.

The present invention regards a tool for use during brain surgery as stated in the preamble of claim 1, and also a system for determining the insertion depth of a probe or similar during brain surgery and the co-ordinates of the tool/probe during brain surgery as stated in the preamble of claim 10.

During brain surgery such as e.g. the implantation of nerve stimulating electrodes for treatment of Parkinson's disease, it is important to insert the electrodes correctly in the first attempt and not least keep the operation as short as possible in order to minimise the stress for the patient, who is awake during the entire procedure.

The known frame-based and frameless conventional stereotactical procedures have several drawbacks, as the procedures are time-consuming, the patient must be transported between the operating room and a CT (Computer Tomography) scanner, discomfort for the patient, difficulties in compensating for target movement (brain displacement).

In the frame-based systems, it is possible that the patient might pull his head out of the frame with the probe inserted in the brain.

The object of the present invention is to avoid the above disadvantages, which object is achieved by a tool of the type mentioned at the beginning, the characteristics of which appear from claim 1, along with a system of the type mentioned at the beginning, the characteristics of which appear from claim 10. Further characteristics of the invention appear from the remaining, dependent claims.

By providing a dynamic reference frame function, it will also be possible to compensate for small movements of the head.

In the following, the invention will be described in greater detail through the use of one possible tool for realising the invention, with reference to the drawings, in which:

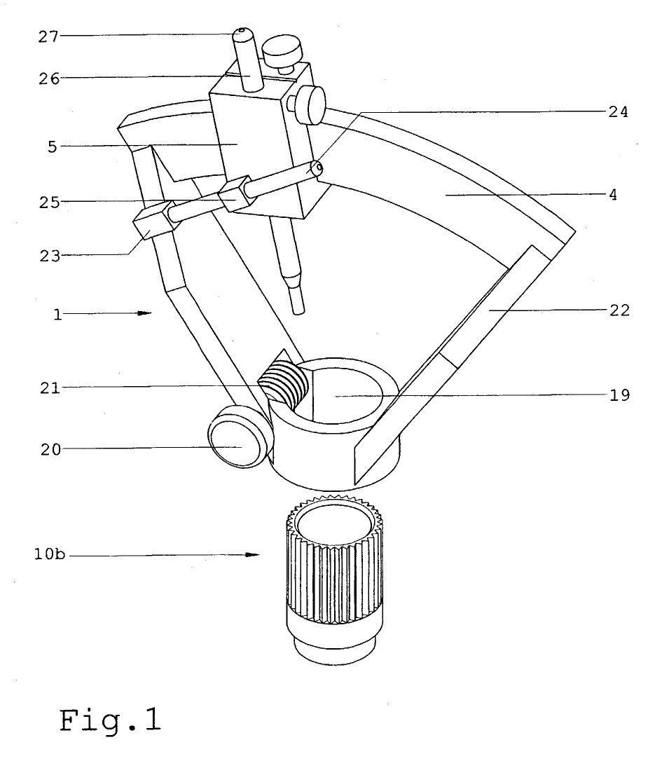

FIG. 1 shows a guide tool with one embodiment of the base part.

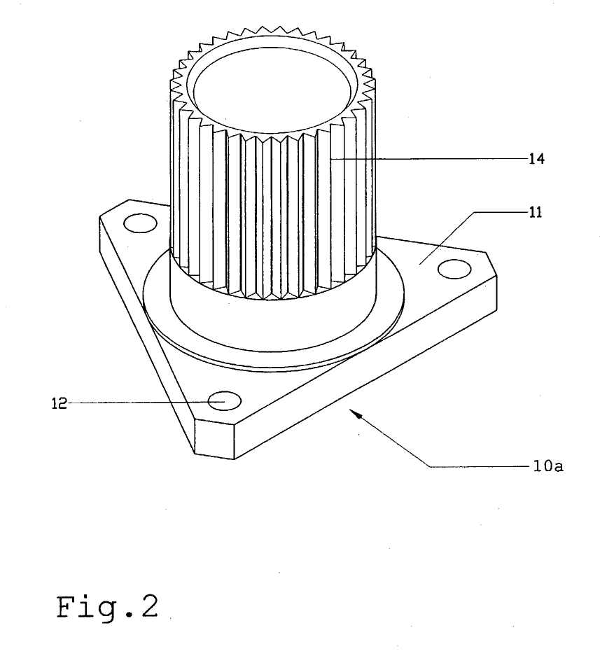

FIG. 2 shows a second embodiment of the base part of the guide tool.

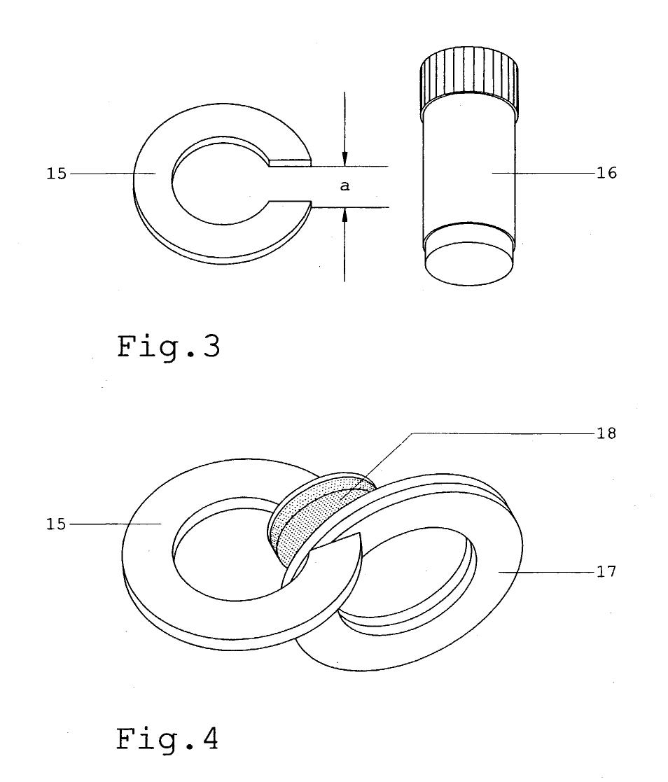

FIG. 3 shows an accessory tool for the guide tool and a plug for the base part.

FIG. 4 shows an application of the accessory tool.

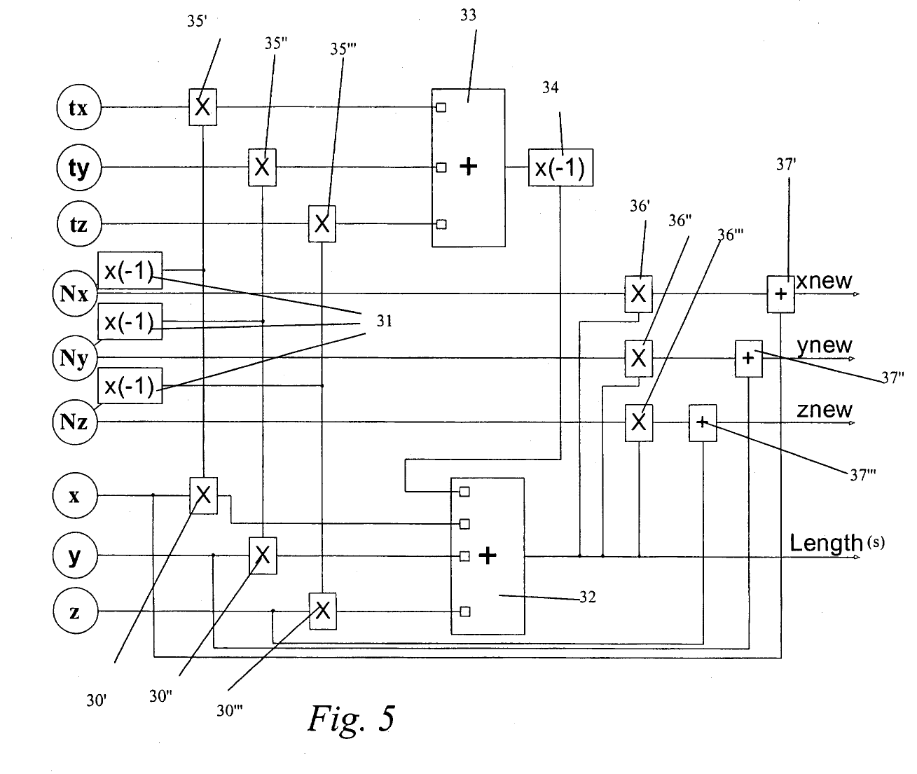

FIG. 5 shows a schematic block diagram of a calculation unit.

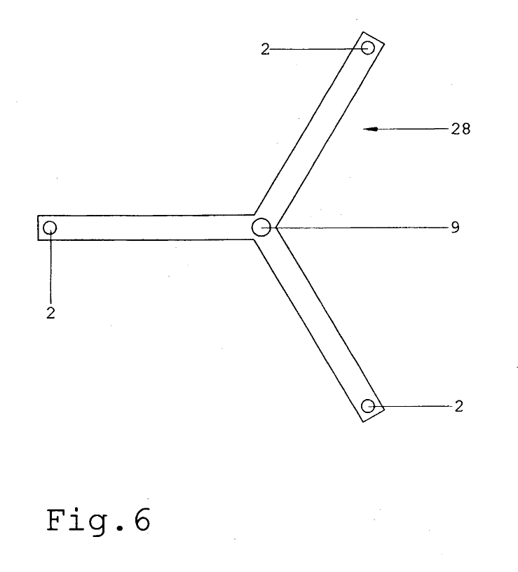

FIG. 6 shows a device for determining the co-ordinates and direction for the guide tool.

The invention is described for application in connection with surgery in a so-called open MR-scanner (Magnetic resonance camera). It will however be possible to use the present invention during surgery in a normal operating room with an image processor connected to a guide/holding tool with a pointer device. The co-ordinates of the image or images of the patient that have been produced in advance in e.g. an MR/CT scanner, refer to anatomical landmarks on the patient's head, such ears, nose etc., or more accurate markers fixed on the skin or in the bone. In case of such an application with reference marks, the reference marks may be placed around a fixing device for the guide/holding tool, which device is arranged on the patient's head (and is described in greater detail below as a base part). The positioning of the guide/holding tool with a pointer device on the patient head is determined by the operation hole in the patient's head, the location of which hole is in turn determined by the type of surgery to be performed, the co-ordinates of said tool being superposed on the image or images displayed by the image processor.

FIG. 1 shows the guide/holding tool 1 for insertion of e.g. a probe into the brain of a patient, which tool is mounted on the patient's head. The guide tool 1 is mounted on one of two base parts 10a, 10b designed to be attached to the patient's head, one 10a by being fastened with three screws 12 that are screwed into the cranium, the other 10b by being screwed straight into the operation hole. The base part 10a, 10b has a hole in the middle, the diameter of which corresponds to the hole drilled in the patient's cranium or is larger than the diameter of the drilled hole. FIG. 3 shows an accessory tool 16 for inserting down into the base part 10a or 10b, for holding the base part 10a, 10b while this is attached to the cranium. The accessory tool 16 is tapered at the end, corresponding to the diameter of the hole drilled in the cranium, while the diameter of the rest of the cylindrical body is equal to the inner diameter of an opening in the base part 10a, 10b. The actual guide part 1 has an annular opening 19 at its base end, the threaded portion 21 of a worm projecting into said opening 19, cf. FIG. 6. The threaded portion co-operates with the knurls 14 on the base part 10, see FIG. 2, so that when the head 20 of the worm is turned, the guide tool 1 will be rotated about the base part 10a, 10b.

It should also be noted that this base part is a disposable piece of equipment and may as mentioned above be equipped with a support plate or supporting arms that project from the base part for positioning of markers as a reference for the co-ordinates of the image or images, which are obtained of the patient in advance in e.g. an MR/CT scanner (the base part then being mounted on the patient's head prior to the MR/CT scanning) instead of anatomical landmarks on the patient's head such as ears, nose etc. or more accurate markers placed on the skin or in the bone. Two types of markers will be provided, or the markers will have two functions; firstly to make marks on the image for positioning the base part and secondly to co-operate with receivers that are placed in defined locations in the room over the site of the operation, and which are connected to a data processing unit connected to an image processing unit in a manner that is known per se. In this way, when taking pictures, marks will be visible on the image for positioning the base part, and in addition the co-ordinates for this position will be registered by the image processing unit, so that the co-ordinates will follow the patient in the case the patient is moved. In this way, the insertion of the probe with its co-ordinates will be calculated by the image processing unit relative to the co-ordinates of the base part and displayed on the image in a manner such that the direction and insertion depth of the probe can be seen by viewing the image. The markers that co-operate with said receivers may be infrared light-emitting diodes, which lights are intercepted by the receivers.

The holder and guide part 5 for the probe is moveable along a curved part 4. The centre point of the radius describing the curved part 4 is located at a distance a below the underside of the base part 10a, 10b. In order for the centre point to lie exactly on the membrane of the brain, the guide/holding tool 1 must be lifted up a certain distance from the scalp by means of washers 17. The thicknesses of the washers are determined by means of an accessory tool 15 (FIG. 3 and FIG. 4) such as e.g. a ring 15 with a slot equal to a. The drilled part 18 of the cranium is placed in the slot, and washers 17 of varying thicknesses are placed in the slot next to the drilled part until the slot is filled. The washers so provided are placed on top of the base part before the guide/holding tool is mounted on this. The slot a is preferably equal to 11 mm, as this slot size is considered to be sufficient to cover individual variations.

An accurate angular adjustment of the guide and fixing of the guide in the desired position may be effected by means of screw 24 with a non-threaded portion at the head, which screw 24 is rotatably supported in a holder that may be rotated about an axis that is perpendicular to the longitudinal direction of the screw 24, and which holder is attached to one end of the curved part 4. The threaded portion of the screw 24 extends into a nut element 25 attached to the guide holder 5, the nut element 25 being rotatable about an axis that is perpendicular to the longitudinal direction of the screw 24. When the screw 24 is rotated, it will rotate freely at 23 and pull or push the guide holder 5 along the curved part. 4. The screw 24 may be provided with a check nut (not shown) for fixing in the desired position, or the holder 5 may be provided with a clamping screw that is screwed down towards the curved part 4, forcing the holder against the curved part 4.

Running through the guide holder 5 is a tube 26 that constitutes the actual guide for a probe, biopsy sampler, laser probe or similar, with the remaining described parts collectively being termed a device for adjusting the direction of the actual insertion tools. The upper part 27 of the tube 26 may have a reduced outer diameter that matches the central aperture 9 of a device 28 for determining the direction of the guide tube 26, the transition from the tube part 26 to the rest of the tube 26 forming an abutment seat for said device 28.

The device 28 includes three arms projecting from its centre, which arms each have a marker 2 at the end. These three markers define a point and a direction that changes only when the holder tool 1 is rotated about the base part 10 or when the holder 5 is moved along the curved part 4.

The markers 2 may be infrared light-emitting diodes, which lights are intercepted by receivers such as 3 linear infrared cameras placed in defined locations above the site of the operation, thus determining the co-ordinates of the insertion tube 26 in a manner that is known per se. The data processor that determines the co-ordinates feeds this information to an image processor.

The markers 2 may also be viewed as markers that may be tracked by a suitable unit such as a camera. The markers may be active and consist of small coils or be passive.

The markers/sensors and the receiving unit may also be of other types than those described herein, the requirement for these units being that they are capable of co-operating with the image processing that is being used.

In order to understand the determination of the direction and the purpose of it, an explanation will be given below of the background for the imaging technique and its application.

Volume data are often used in industry, in the military and within the field of medicine. Volume data is taken to mean large quantities of imagery to be processed; one example from the field of medicine is sectional views of the human body, where a number of pictures are taken in section through the human body, also in several planes. Innumerable different image systems are capable of collecting volume data. What they all have in common is that they are based on emitted radiation/energy that can penetrate the surface of the object being represented. Examples of such radiation/energy are ultrasound, X-rays, MRI (electromagnetic radiation) and infrared light.

Volume data can not be shown directly, as the human vision is based on stereo vision, the eyes acting as two two-dimensional cameras. Volume data must therefore be processed before it can be comprehended by the human brain.

In spite of great efforts to develop visualisation methods, the method most used for reading volume data in the medical field up until now, is to view two-dimensional cross sections cut out of a volume with a limited slice thickness. In medicine, three standard slice directions are defined relative to the orientation of the patient (axial, sagittal and coronal). These planes are orthogonal.

When information regarding a structure underneath the surface of an object is of interest, three-dimensional imaging may provide this information. In order to be able to relate information from volume data to the physical object, the physical space and image space must be correlated.

Visualisation of three-dimensional volume data and correlation of this to the object in the physical space is performed in a large number of applications, including neurosurgery. Several commercial so-called neuro-navigation systems are known which allow the above within the field of surgery. These systems are composed of a computer, a camera and a pointer device. The pointer device can trace in three-dimensional space to allow its position and direction to be calculated/determined. The previously mentioned arms 28 with markers 2 constitute such a pointer device. The pointer device may thus be used to register the physical space of the image space by identifying known points both physically and in the image, and for interactive navigation in the image space.

All commercial neuro-navigation systems available at present have two modes that are used for navigation through image data. The first mode, perhaps the most common, visualises three orthogonal planes (axial, sagittal and coronal) with the position of the pointer device as a common point in the three planes. In the second mode, one plane is visualised perpendicular to the pointer device and another plane in the plane of the pointer device.

The available visualisation modes are normally easy to relate to but may be insufficient in applications where a target must be hit/reached with great precision, as in the case of stereotactical surgical procedures. In the case of such applications, decoupling the degrees of freedom in the movement is advantageous in that the operator does not have to relate to all of the degrees of freedom at the same time, while also having to see the target.

Based the above, a method has been found in which the length of the pointer device, i.e. the insertion tube, is varied logically (virtually) so that a perpendicular plane through the logical tip always contains the target. One possible way of effecting this is to introduce a further level in the calculation of the co-ordinates of the pointer device and in the visualisation routines.

The purpose of the length determination is also to be able to provide a measurement for how far e.g. a probe is to be inserted into the brain before it reaches the target. The distance s from the target is determined by the position (the co-ordinates) of the tip of the pointer device and the co-ordinates of the target, determined in a manner that is known per se, with these parameters being supplied to a calculation unit that solves the following equations:

s=nₓ(xₜ−x₀)+n_{y}(yₜ−y₀)+n_{z}(zₜ−z₀)

xₗ=nₓ·s+x₀

yₗ=n_{y}·s+y₀

zₗ=n_{z}·s+z₀

which can be expressed by a linear algebra as:

\vec{x}ₗ=\vec{n}^{T}·(\vec{x}ₜ−\vec{x}₀)·\vec{n}+\vec{x}₀

in which T=target, 0=physical position of the pointer device, l=logical/calculated position, x, y, z, is the position of the pointer device, n indicates the direction of the pointer device and n with the indices x, y, z gives the direction in the x, y, and z directions respectively.

Introducing the additional logical level allows decoupling of the movement, which simplifies the adjustment of a pointer device of the type mentioned previously considerably, so as make it hit a defined target. By visualising a perpendicular plane on the pointer device, which plane contains the target at all times, the pointer device (with two degrees of freedom) being adjustable so that the path of the pointer device passes through the target. This plane is produced in the image processor by replacing the physical position of the pointer device with the logical one, as described above. When the direction is locked, the distance (depth), i.e. the distance from the pointer device to the target, may be calculated by the equation as described above, in which s is the distance, this calculation being performed by means of the system shown by the schematic block diagram in FIG. 8.

The position of the pointer device has been determined by a method that is known per se, and these values x, y, z are fed to respective multipliers 30′, 30″, 30″ in which they are multiplied by the sign-inverted value 31 directions of the respective direction of the pointer device nx, ny, nz, which direction has been determined in a manner that is known per se, and which is then added in an adder 32 together with the sign-inverted value 34 of the sum of from another adder 33. The sum from the further adder 33 results by said sign-inverted values 31 of the direction of the pointer device being multiplied in respective multipliers 35′, 35″, 35′″ by respective target co-ordinates and fed to the further adder 33 for adding. The result from the adder 32 equals the length s, i.e. an extension of the pointer device, which in turn will be a measure of how far e.g. a probe is to be inserted. Further, it will also be possible to provide a modified position for the tip of the pointer device, a virtual extension. The co-ordinates of the modified position are in FIG. 8 denoted x new, y new and z new. As appears from FIG. 8, these parameters are produced by the respective values of the direction Nx, Ny, Nz of the pointer device being multiplied in respective multipliers 36′, 36″, 36′″ by the length s, and the values thus produced are added in the respective adders 37′, 37″, 37′″ to the respective positions x,y,z, whereby the modified positions xnew, ynew, znew are obtained. The co-ordinates thus obtained are used for interactive navigation in a recording of a volume of e.g. a brain to be operated on. The position and direction of the pointer device is visualised on a display screen along with sectional images from the volume. This allows the surgeon in an image-guided manner to adjust the pointer device so that this is pointing exactly at the intended target. The depth to the target is calculated, and the probe may be inserted into the insertion tube. The probe may be marked or provided with a stop located on the probe with respect to the tip of the probe and the length of insertion determined by the above method.

The accuracy of the system has been tested on a model of a head made of plastic and filled with gelatine mixed with CuSO₄. The model head was fixed with a 3 point Mayfield clamp, and a flexible surface coil was positioned for intraoperative imaging. 23 different virtual targets were tested, all of which could be reached with a glass needle through a 16 mm drilled hole in the model head.

The above described tool was mounted on the model head, after which a three-dimensional MR image was obtained. The target was identified from this image, the tool was adjusted in order to be able to reach the target (with the virtual extension of the tip), and the depth (distance s) was calculated. The insertion depth of the glass needle was adjusted to the calculated length by means of a stop collar at one end of the needle, and the needle was inserted during continuous MR imaging. Two MR images were produced following each hit at the intended target. The images were obtained in two orthogonal planes, both with the needle in the plane. The position of the tip of the needle was taken as an average of the positions found in the two planes. It should be noted that the procedure carried out on the model head was the same as that which would be carried out on a patient. All targets were reached with an average error of 0.7 mm and a maximum error of 1.3 mm. The pixel size used was 0.97 mm, and as such the error must be considered to be of the order of that which lies in the discretisation process, which is inherent in the nature of the MR (as this is a digital imaging technique) and thus comparable to that of optimal frame based systems.

The total time taken from identification of the target co-ordinates to the final verification of the needle position was approximately 15 minutes.

Based on the above, is should be obvious that the present invention overcomes many of the problems associated with the systems that are in use today. Thus the problems associated with target movement after opening of the cranium and inaccuracies introduced by converting from an MR/CT room to a stereotactical room are eliminated. Moreover, this allows a direct verification of the probe's position in the brain, which allows any repositioning required in the case of error.

1. A tool for use during brain surgery, where a guide/holding tool (1) is designed to be mounted on the patient's head over an operation hole in a manner such that e.g. a probe may be inserted into a patient's brain, characterised in that the probe is held in a holder and guide part (5) in a manner that is known per se, which holder and guide part (5) is arranged slidably along a curved part (4) in the upper part of the tool (1),
that the centre point of the radius of the curved part (4) is located at a distance (a) below the underside of the base part (10a, 10b),
that the tool includes means designed to adjust the positioning of the centre point, i.e. the distance (a) of the centre point from the underside of the base part (10a, 10b), and
that the base part (10a, 10b) and the probe are equipped with markers for determining the co-ordinates of the respective parts for display in an image.

2. A tool according to claim 1, characterised in that said tool (1) includes an attachment for a pointer device formed by a guide tube (26) with an upper part (27) for attachment of signal transmitters for calculation of the direction and co-ordinates of the pointer device, which signal transmitters are connected to an image processing unit for calculation of the insertion depth of the probe, the co-ordinates and direction of the probe/guide tool being superposable on the visualised image or images of the brain, and
that said tool (1) includes adjustment means (7; 21) for adjustment of the tool (1) to the desired direction of the probe.

3. A tool according to claim 1, characterised in that the means of adjusting said distance (a) include washers (17) designed to be placed between the base part (10a, 10b) and the curved part (4) in a manner such that the centre point of the radius is lifted up and lies exactly on the membrane of the brain.

4. A tool according to claims 1-4, characterised in that it comprises an accessory tool (15) for determining the thickness of the washers (17), which tool (15) is formed by a ring (15) with a slot having an extent equal to said distance (a), said distance (a) being equal to the drilled part (18) of the cranium in the operation hole plus washers (17).

5. A tool according to claims 1-5, characterised in that the holder and guide part (5), with the curved part (4) in the upper part of the tool (1), is arranged to be rotatable about the base part (10a, 10b).

6. A tool according to claims 1-6, characterised in that a tube (26) extends through the holder and guide part (5), which tube is the actual guide for a probe, biopsy sampling tool, laser probe or similar, that the upper part (27) of the tube (26) has a reduced diameter (27) that matches the central aperture (9) of a device (28) for determining the direction of the guide tube (26), the transition from the part of the tube to the remaining part of the tube (26) forming an abutment seat for said device (28), and that the device (28) includes means of determining the direction and position of the guide tube (26).

7. A tool according to claims 1-7, characterised in that the device (28) incorporates three arms projecting from the centre of the device, each of which arms have a marker (2) at the end, and that the device (28) is held in a specific position on the holder and guide part (5), so that the position calculated from the markers is changed only when the holding tool (1) is rotated about the base part (10) or when the holder and guide part (5) is moved along the curved part (4).

8. A tool according to claims 1-8, characterised in that the marker (2) consists of infrared light-emitting diodes, which lights are intercepted by receivers located in defined positions in the room over the site of the operation, thereby to determine the co-ordinates of the insertion tube (26) in a data processor connected to an image processor, in a manner that is known per se.

9. A base part for a tool for use during brain surgery, where a guide/holding tool (1) is designed to be mounted on the patient's head over an operation hole in such a manner that e.g. a probe may be inserted into a patient's brain, characterised in that the base part (10a, 10b) includes holes for screwing to the patient's cranium or is designed to screwed down into the operation hole or designed to be glued to the patient's cranium,
that the base part includes a support plate or supporting arms projecting from the base part for placement of marker, there being provided two types of markers or markers with two functions; firstly to make marks on the image for positioning the base part and secondly to co-operate with receivers that are placed in defined locations in the room over the site of the operation, and which are connected to a data processing unit connected to an image processing unit in a manner that is known per se, as a reference for the co-ordinates of the image or images obtained in advance of the patient, e.g. in an MR/CT scanner, and
that the opening in the base part for insertion of the probe is approximately the same size as the operation hole or at least large enough to make it possible to see into the operation hole during insertion of the probe.

10. A system for determining the insertion depth of a probe or similar during brain surgery and the co-ordinates of the probe/tool for use during brain surgery, characterised in that a virtual length of an insertion tube or pointer device is visualised in an image processor, so that a perpendicular plane through the logical tip of the insertion tube always contains the target, i.e. the area of the brain that is of interest, and that the co-ordinates that are determined in a manner that is known per se in the image processor, are fed to a calculation unit for determining the insertion depth by the equation
s=nₓ(xₜ−x₀)+n_{y}(yₜ−y₀)+n_{z}(zₜ−z₀)
in which:
xₗ=nₓ·s+x₀
yₗ=n_{y}·s+y₀
zₗ=n_{z}·s+z₀
which can be expressed by means of a linear algebra as:
\vec{x}ₗ=\vec{n}^{T}·(\vec{x}ₜ−\vec{x}₀)·\vec{n}+\vec{x}₀
in which T=target, 0=physical position of the pointer device, l=logical/calculated position, x, y, z, is the position of the pointer device, n indicates the direction of the pointer device and n with the indices x, y, z indicates the direction in the x, y, and z directions respectively.

11. A system according to claim 10, characterised in that the calculation unit comprises a first set of multipliers (30′, 30″, 30″), the input signal to which is the position (x, y, z) of the pointer device, determined in advance in a manner that is known per se, and the sign-inverted values (31) of the direction of the respective direction of the pointer device(nx, ny, nz), which is already determined in a manner that is known per se, where the output of the first set of multipliers (30′, 30″, 30″) is connected to an adder (32), which has a further input connected to the output of a sign-inverter (34), the input to which is connected to the output of a further adder (33), where the input to the further adder (33) is connected to the output of the respective of a second set of multipliers (35′, 35″, 35′″), where the input to the second set of multipliers (35′, 35″, 35′″) is fed to respective target co-ordinators (tx, ty, tz) and the respective sign-inverted values (31) of the direction of the direction of the pointer device (nx, ny, nz), which is already determined in a manner that is known per se, so that the output signal from the further adder (32) equals the length(s), i.e. an extension of the pointer device, which in turn will be a measure of how far e.g. a probe is to be inserted, and which is processed in an image processor for display on a screen together with one or more images of a patient's brain.

12. A system according to claims 10-11, characterised in that a modified position of the tip of the pointer device, a virtual extension, is provided by said length(s) being fed to a third set of multipliers (36′36″, 36″′), a second input to the third set of multipliers (36′ 36″, 36′″) is fed the respective values of the direction (Nx, Ny, Nz) of the pointer device, and the output from the respective third set of multipliers (36′ 36″, 36′″) is connected to the inputs to a set of adders (37′, 37″, 37″′), which has a further input connected to the values (x,y,z), so that the output signal from the set of adders (37′, 37″, 37′″) is the modified positions (xnew, ynew, znew).
